(19) United States
(12) Patent Application Publication
(10) Pub. No.: US 2022/0161000 A1
(43) Pub. Date: May 26, 2022

(54) MULTI-DUROMETER ATTACHMENT DEVICE FOR CHRONIC FIXATION

(71) Applicant: Medtronic, Inc., Minneapolis, MN (US)

(72) Inventors: Jason D. Hamack, Ramsey, MN (US); Pankti N. Shah, Minneapolis, MN (US); Scott N. Tuominen, Colombia Heights, MN (US)

(21) Appl. No.: 17/102,807

(22) Filed: Nov. 24, 2020

(51) Int. Cl. A61M 25/02

(52) U.S. Cl. CPC A61M 25/02; A61M 2025/0286

(57) ABSTRACT

An attachment device for securing a medical device to a patient, comprising at least one layer including a flexible portion and a rigid portion. The rigid portion being connected to the flexible portion and defining at least one loop. The at least one loop including an elongate body and an anchoring element. The elongate body having a first end and a second end opposite the first end. The anchoring element being disposed circumferentially around, and connected to, the elongate body.

### CROSS-REFERENCE TO RELATED APPLICATION

n/a.

### FIELD

The present technology is generally related to attachment devices for medical devices.

### BACKGROUND

Many routine clinical treatments involve the percutaneous and transcutaneous insertion of medical systems into the human body. One challenge clinicians often face is ensuring that a medical device can be permanently attached and positioned on a patient's body over a period of time without causing harm to the patient. Existing methods require the use of heavy attachment devices to couple the medical device to the body of the patient. This is performed by connecting the medical device to the attachment device and then mounting the attachment device on the patient's body. However, attachment devices are often ill-suited for patients who seek the ability to move freely throughout the day. For example, without the use of multi-durometer materials, the attachment device is often rigid and causes discomfort to the patient when they move or contort their body a certain way. Additionally, because attachment devices are heavy, they often lead to tearing of the patient's skin due to the combined weight of the medical device and the attachment device.

### SUMMARY

The techniques of this disclosure generally relate to devices used for facilitating the attachment of a medical device to a patient.

In one aspect, the present disclosure provides an attachment device for securing a medical device to a patient, comprising at least one layer including a flexible portion and a rigid portion. The rigid portion being connected to the flexible portion and defining at least one loop. The at least one loop including an elongate body and an anchoring element. The elongate body having a first end and a second end opposite the first end. The anchoring element being disposed circumferentially around, and connected to, the elongate body.

In another aspect of this embodiment, the flexible portion and the rigid portion are each composed of a mixture of a flexible first material and a rigid second material, the flexible first material having a lower durometer than the rigid second material.

In another aspect of this embodiment, the mixture includes a higher concentration of the flexible first material than the rigid second material in the flexible portion and the mixture includes a higher concentration of the rigid second material than the flexible first material in the rigid portion.

In another aspect of this embodiment, the first end defines a first aperture and the second end defines a second aperture.

In another aspect of this embodiment, the at least one loop includes a longitudinal axis extending between the first end and the second end, the first end and the second end defining a channel therebetween sized to receive a suturing element.

In another aspect of this embodiment, the first aperture has a first diameter and the second aperture has a second diameter, the second diameter being different from the first diameter.

In another aspect of this embodiment, the anchoring element includes a first end, a second end opposite the first end, and a lateral surface therebetween.

In another aspect of this embodiment, the elongate body is tapered inwardly along the longitudinal axis from the first end to the second end.

In another aspect of this embodiment, the first end of the anchoring element is proximate to the first end of the elongate body and the second end of the anchoring element is proximate to the second end of the elongate body.

In another aspect of this embodiment, the anchoring element and the elongate body define a gap therebetween.

In another aspect of this embodiment, the flexible portion has a Shore A hardness level between the range of 30 and 50.

In another aspect of this embodiment, the rigid portion has a Shore A hardness level between the range of 80 and 95.

In another aspect of this embodiment, the at least one layer includes a plurality of layers, the plurality of layers being connected to form a housing sized to retain the medical device therein.

In one aspect, a suture loop comprising an elongate body having a first end and a second end opposite the first end. A longitudinal axis extending between the first end and the second end of the elongate body. The first end and the second end defining a channel along the longitudinal axis sized to receive a suturing element. An anchoring element disposed circumferentially around, and connected to, the elongate body. The anchoring element having a first end, a second end opposite the first end, and a lateral surface therebetween.

In another aspect of this embodiment, the first end defines a first aperture and the second end defines a second aperture.

In another aspect of this embodiment, the first aperture has a first diameter and the second aperture has a second diameter, the second diameter being different from the first diameter.

In another aspect of this embodiment, the elongate body is tapered inwardly along the longitudinal axis from the first end to the second end.

In another aspect of this embodiment, the first end of the anchoring element is proximate to the first end of the elongate body and the second end of the anchoring element is proximate to the second end of the elongate body.

In another aspect of this embodiment, the elongate body and the anchoring element have a Shore A hardness level between the range of 80 and 95.

In one aspect, an attachment device, comprising: at least one layer, the at least one layer including a flexible portion having a Shore A hardness level between the range of 30 and 50 and a rigid portion having a Shore A hardness level between the range of 80 and 95. The flexible portion and the rigid portion each being composed of a mixture of a flexible first material and a rigid second material, the flexible first material having a lower durometer than the rigid second material. The mixture including a higher concentration of the flexible first material than the rigid second material in the flexible portion and the mixture including a higher concentration of the rigid second material than the flexible first material in the rigid portion. The rigid portion defining at least one suture loop including an elongate body having a first end, a second end opposite the first end, and a longitudinal axis extending therebetween. The first end defining a first aperture and the second end defining a second aperture, the first aperture having a diameter greater than the second aperture. The elongate body being tapered inwardly along the longitudinal axis from the first end to the second end and defining a channel therebetween sized to receive a suturing element. The at least one suture loop further including an anchoring element disposed circumferentially around the elongate body. The anchoring element including a first end, a second end opposite the first end, and a lateral surface therebetween. The first end of the anchoring element being proximate to the first end of the elongate body, the second end of the anchoring element being proximate to the second end of the elongate body. The anchoring element and the elongate body defining a gap therebetween.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 is a sectional view of single layer of material of an attachment device for securing a medical device to a patient;

FIG. 2 is a sectional view of the attachment device of FIG. 1, wherein multiple layers are combined to form a housing;

FIG. 3 is a perspective view of the attachment device of FIGS. 1 and 2;

FIG. 4 is a perspective view of a suturing loop defined by each layer of the attachment device of FIGS. 1 and 2;

FIG. 5 is a sectional view of the suturing loop of FIG. 4; and

FIG. 6 is a sectional view of the attachment device of FIGS. 1-4, which illustrates the connection between the flexible first material and the suturing loops within each layer.

### DETAILED DESCRIPTION

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

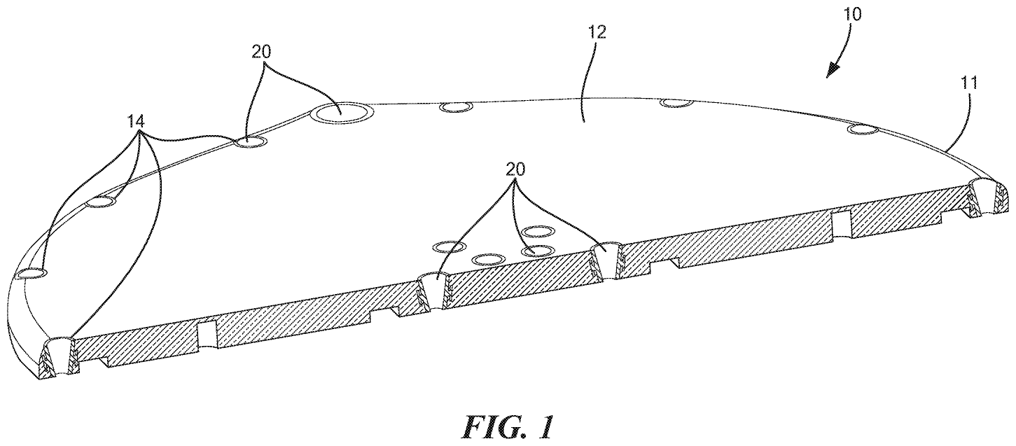

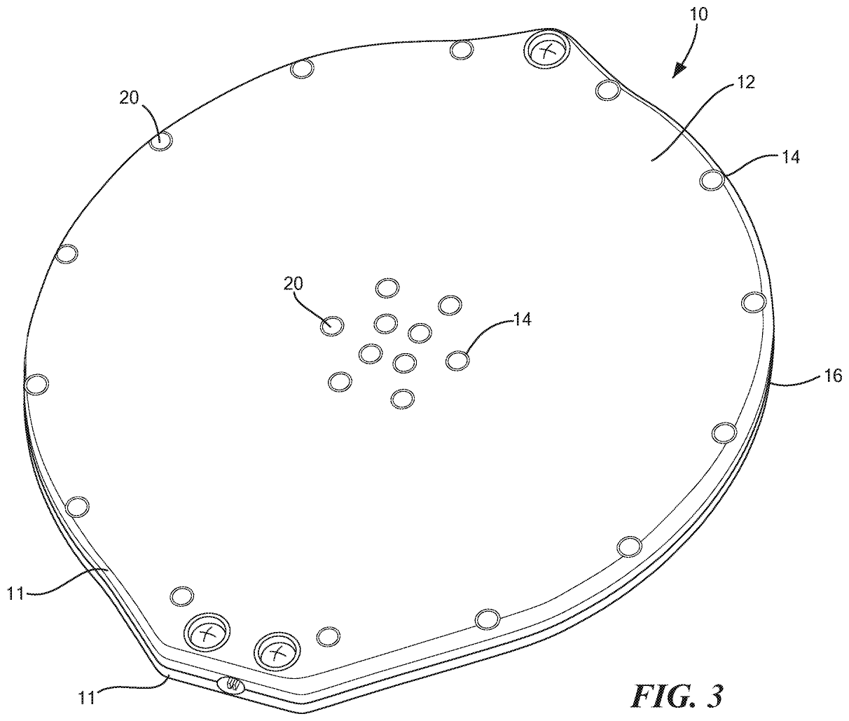

Referring now to the drawings in which like reference designators refer to like elements there is shown in FIGS. 1-3 an exemplary attachment device 10 for securing a medical device to a patient. The device 10 includes at least one layer 11 made up of a flexible portion 12 and a rigid portion 14. The flexible portion 12 has a Shore A hardness level between the range of 30 and 50, and the rigid portion 14 has a Shore A hardness level between the range of 80 and 95.

The flexible and rigid portions 12, 14 of each layer 11 are each made up of a mixture of a flexible first material and a rigid second material which are blended together at varying concentrations to allow for a desired durometer level to be achieved. For example, when mixed, if the concentration of the rigid second material is higher than the concentration of the flexible first material, the resulting layer 11 will have a higher durometer. Thus, the rigid portion 14 of each layer 11 is made up of a mixture having a higher concentration of the rigid second material than the flexible first material. Conversely, the flexible portion 12 of each layer 11 has a lower durometer and is made up of a mixture having a higher concentration of the flexible first material than the rigid second material. In embodiments with more than one layer 11, there may be numerous combinations of layers having a higher durometer and/or a lower durometer.

Further, each material that is mixed to form each layer 11 of the attachment device 10 is any material that could be used in an additive manufacturing processes that would allow for multiple durometers to be applied to the same part in the same build. In an exemplary embodiment, the flexible first material may be Agilus or other like flexible UV Cure Resins and materials having similar flexibility and the rigid second material 14 may be Vero or other like rigid UV Cure Resins and materials having similar rigidity. When the flexible first material and the rigid second material are blended together, the resulting mixture may have a Shore A hardness level between the range of 30 and 95 depending on the concentration of each material when mixed.

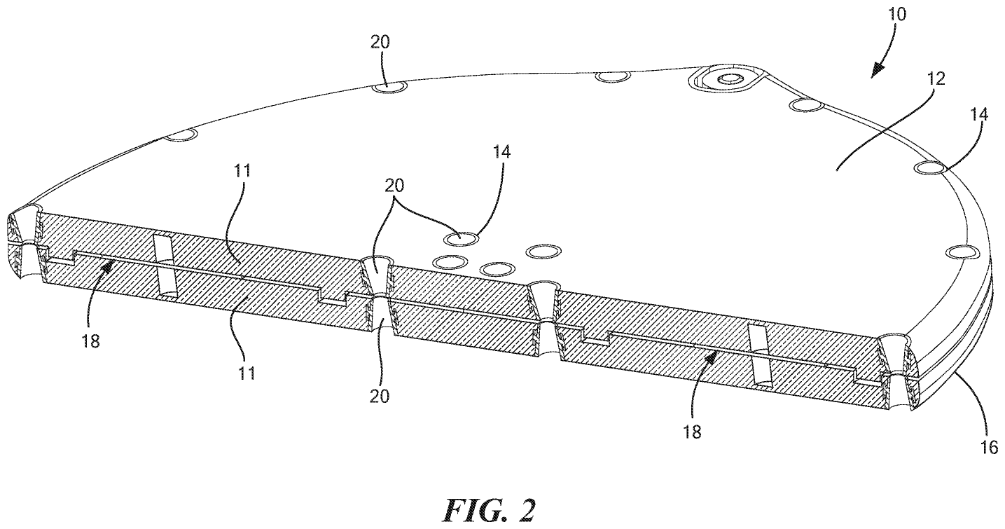

Referring now to FIG. 2, multiple layers 11 of the device 10 may be combined to form a housing 16 defining a cavity 18 therein sized to retain a medical device. Once a medical device is disposed within the cavity 18, the layers 11 of the device 10 are bounded together with a medical adhesive or connector, such as a screw, and may undergo a curing process. In an exemplary embodiment the housing 16 may be sized to retain an external transmission coil for a transcutaneous energy transfer system (TETS). However, the device 10 may be sized to facilitate the attachment of various other treatment devices to the body of a patient.

As shown in FIGS. 1-3, the rigid portion 14 may define at least one suturing “loop,” “hole,” or any other similar opening 20 disposed throughout the at least one layer 11. Each suturing loop 20 may have a different diameter and depth to accommodate the use of suture needles and threads of various sizes. Each loop 20 may be permanently integrated within the flexible portion 12 or may be configured to be readily inserted and removable from the flexible portion 12. This allows for each loop 20 to be replaced or repaired if it becomes damaged or defective. Further, each loop 20 may define a channel sized to receive the insertion of a suturing needle, thread, or the like so that the device 10 may be sutured, mounted, or otherwise attached to the patient. The flexibility of the flexible portion and the rigidity of the rigid portion 14 provides increased comfort to the patient while still ensuring that the device 10 is securely attached to the patient. The combination of the flexible portion 12 and the rigid portion 14 in each layer 11 allows the device 10 to readily contort and match the curvature of the patient's body as the patient moves throughout the day, thus reducing the risk of tearing or damage of the patient's skin. The rigid portion 14 also provides greater retention strength so that the suture loops 20 and the device 10 do not become detached from the patient as the patient moves.

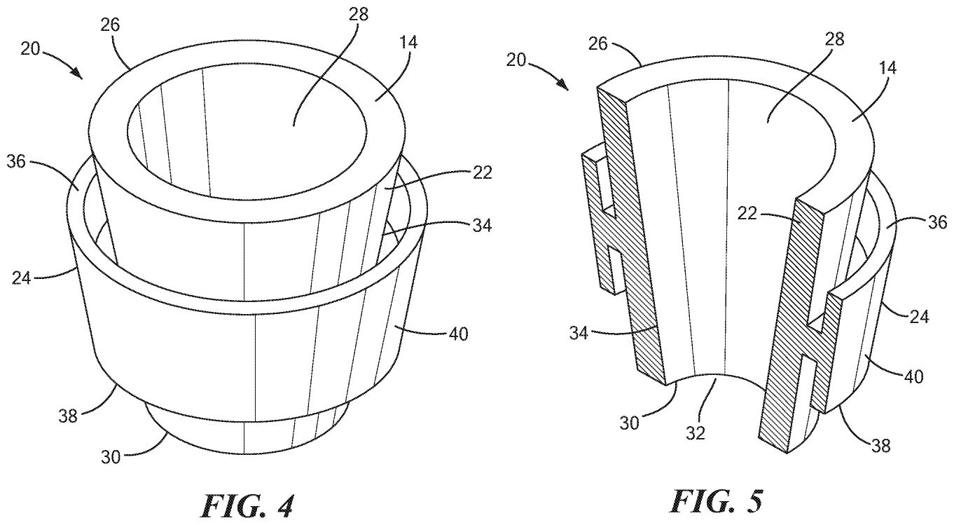

Now referring to FIGS. 4-6, each suturing loop 20 may include an elongate body 22 and an anchoring element 24. Alternatively, the suturing loops 20 may not include an elongate body 22 and may instead lie entirely within a singular plane. The elongate body 22 of the loop 20 includes a first end 26 defining a first aperture 28, a second end 30 defining a second aperture 32, and a lateral surface 34 extending therebetween along a longitudinal axis defined by the first end 26 and second end 30. The first aperture 28 defines a first diameter and the second aperture 32 defines a second diameter, the second diameter being equal to or different from the first diameter. In embodiments where the diameter of the first aperture 28 is larger than the diameter of the second aperture 32, the lateral surface 34 tapers inwardly along the longitudinal axis from the first end 26 to the second end 30. However, in embodiments where the diameter of the second aperture 32 is greater than the diameter of the first aperture 28, the lateral surface 34 may taper outwardly along the longitudinal axis from the first end 26 to the second end 30. In embodiments where the diameter of the second aperture 32 is equal to the diameter of the first aperture 28, the lateral surface 34 does not taper along the longitudinal axis.

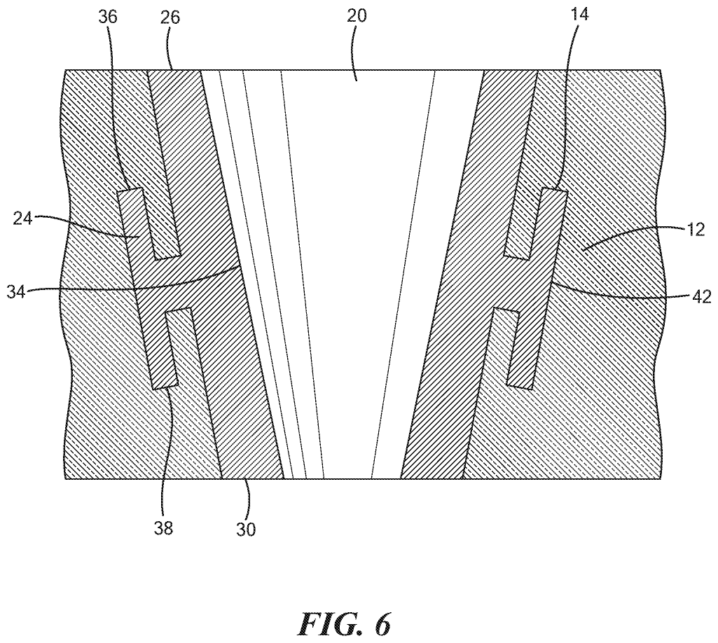

Continuing to refer to FIGS. 4-6, the anchoring element 24 may be a hook, clip, or other type of attachment member that is disposed circumferentially around and connected to the elongate body 22. The anchoring element 24 includes a first end 36, a second end 38 opposite the first end 36, and a lateral surface 40 therebetween. The first end 36 of the anchoring element 24 is proximate to the first end 26 of the elongate body 22 and the second end 38 of the anchoring element 24 is proximate to the second end 30 of the elongate body 22. The first and second ends 36, 38, each may be a first and second lip portion, respectively. As shown in FIG. 6, when the flexible first material 12 and the rigid second material 14 are connected, the anchoring element 24 is received within a receiving portion 42 defined by the flexible first material 12 so that the flexible first material 12 and rigid second material 14 are interlocked with each other and the lateral surface 40 of the anchoring element 24 is flush with the flexible first material 12. Further, the anchoring element 24 and the elongate body 22 may define a groove 44 therebetween to allow for slight flexibility and movement of the elongate body 22. However, in alternate embodiments there may be no groove 44 between the anchoring element 24 and the elongate body 22.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention, which is limited only by the following claims.

What is claimed is:

1. An attachment device for securing a medical device to a patient, comprising:
at least one layer including:
a flexible portion; and
a rigid portion, the rigid portion being connected to the flexible portion and defining at least one loop, the at least one loop including:
an elongate body, the elongate body having a first end and a second end opposite the first end; and
an anchoring element being disposed circumferentially around and connected to the elongate body.

2. The device of claim 1, wherein the flexible portion and the rigid portion are each composed of a mixture of a flexible first material and a rigid second material, the flexible first material having a lower durometer than the rigid second material.

3. The device of claim 2, wherein the mixture includes a higher concentration of the flexible first material than the rigid second material in the flexible portion and the mixture includes a higher concentration of the rigid second material than the flexible first material in the rigid portion.

4. The device of claim 3, wherein the first end defines a first aperture and the second end defines a second aperture.

5. The device of claim 4, wherein the at least one loop includes a longitudinal axis extending between the first end and the second end, the first end and the second end defining a channel therebetween sized to receive a suturing element.

6. The device of claim 5, wherein the first aperture defines a first diameter and the second aperture defines a second diameter, the second diameter being different from the first diameter.

7. The device of claim 6, wherein the anchoring element includes a first end, a second end opposite the first end, and a lateral surface therebetween.

8. The device of claim 7, wherein the elongate body is tapered inwardly along the longitudinal axis from the first end to the second end.

9. The device of claim 8, wherein the first end of the anchoring element is proximate to the first end of the elongate body and the second end of the anchoring element is proximate to the second end of the elongate body.

10. The device of claim 9, wherein the anchoring element and the elongate body define a gap therebetween.

11. The device of claim 10, wherein the flexible portion has a Shore A hardness level between the range of 30 and 50.

12. The device of claim 11, wherein the rigid portion has a Shore A hardness level between the range of 80 and 95.

13. The device of claim 12, wherein the at least one layer includes a plurality of layers, the plurality of layers being connected to form a housing sized to retain the medical device therein.

14. A suture loop, comprising:
an elongate body having:
a first end and a second end opposite the first end;
a longitudinal axis extending between the first end and the second end of the elongate body, the first end and the second end defining a channel along the longitudinal axis sized to receive a suturing element; and
an anchoring element disposed circumferentially around and connected to the elongate body, the anchoring element having a first end, a second end opposite the first end, and a lateral surface therebetween.

15. The suture loop of claim 14, wherein the first end of the elongate body defines a first aperture and the second end of the elongate body defines a second aperture.

16. The suture loop of claim 15, wherein the first aperture has a first diameter and the second aperture has a second diameter, the second diameter being different from the first diameter.

17. The suture loop of claim 16, wherein the elongate body is tapered inwardly along the longitudinal axis from the first end to the second end.

18. The suture loop of claim 17, wherein the first end of the anchoring element is proximate to the first end of the elongate body and the second end of the anchoring element is proximate to the second end of the elongate body.

19. The suture loop of claim 18, wherein the elongate body and the anchoring element are made up of a rigid portion having a Shore A hardness level between the range of 80 and 95.

20. An attachment device, comprising:
at least one layer, the at least one layer including a flexible portion having a Shore A hardness level between 30 and 50 and a rigid portion having a Shore A hardness between the level of 80 and 95, the flexible portion and the rigid portion each being composed of a mixture of a flexible first material and a rigid second material, the flexible first material having a lower durometer than the rigid second material, the mixture including a higher concentration of the flexible first material than the rigid second material in the flexible portion and the mixture including a higher concentration of the rigid second material than the flexible first material in the rigid portion; and
the rigid portion defining at least one suture loop including:
an elongate body having a first end and a second end opposite the first end, the first end defining a first aperture and the second end defining a second aperture, the first aperture having a diameter greater than the second aperture;
a longitudinal axis extending between the first end and the second end, the elongate body being tapered inwardly along the longitudinal axis from the first end to the second end and defining a channel therebetween sized to receive a suturing element; and
an anchoring element disposed circumferentially around the elongate body, the anchoring element including a first end, a second end opposite the first end, and a lateral surface therebetween, the first end of the anchoring element being proximate to the first end of the elongate body, the second end of the anchoring element being proximate to the second end of the elongate body, the anchoring element and the elongate body defining a gap therebetween.
